(19) United States
(12) Patent Application Publication
(10) Pub. No.: US 2010/0026958 A1
(43) Pub. Date: Feb. 4, 2010

(54) Fitting Method for Multifocal Lenses

(76) Inventors: C. Benjamin Wooley, Jacksonville, FL (US); Susan W. Neadle, Jacksonville, FL (US); Thomas R. Karkkainen, Orange Park, FL (US); Ronald J. Clark, Jacksonville, FL (US); Amitava Gupta, Roanoke, VA (US); Sheila B. Hickson-Curran, Ponte Vedra, FL (US); Gregory A. Tatarka, Jacksonville, FL (US); Arkady Selenow, Irvington, NY (US); George Zikos, New York, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/185,192

(22) Filed: Aug. 4, 2008

(51) Int. Cl. A61B 3/18

(52) U.S. Cl. 351/247

(57) ABSTRACT

The invention provides methods for fitting multifocal contact lenses that are less time consuming and result in more successful fittings than conventional methods.

### FIELD OF THE INVENTION

The invention relates to fitting ophthalmic lenses useful for the correction of presbyopia. In particular, the invention provides methods for fitting multifocal contact lenses to correct presbyopia.

### BACKGROUND OF THE INVENTION

As an individual ages, the eye is less able to accommodate, or bend the natural lens, to focus on objects that are relatively near to the observer. This condition is known as presbyopia. Similarly, for persons who have had their natural lens removed and an intraocular lens inserted as a replacement, the ability to accommodate is absent.

Among the methods used to correct for the eye's failure to accommodate is a method known as mono-vision in which a single vision lens for correction of distance vision is used in the lens wearer's dominant eye and a single vision lens for correction of near vision is used in the non-dominant eye. Another known method for correction of presbyopia is to use bifocal or multifocal contact lenses in both of the individual's eyes. Yet another method of treating presbyopia is to place a bifocal or multifocal lens in one eye and a single vision lens in the other eye.

Regardless of the correction method used, successful fitting of the lenses using conventional methods is a function of trial and error. Typically, once the individual is refracted to determine the visual correction required, a set of trial lenses will be used by the eye care practitioner in a search for the highest level of visual comfort when viewing a standard visual target. Among the disadvantages in using this method to fit multifocal lenses is that the simultaneous images presented to the lens wearer's retinas requires that the effect of image blur be minimized. To minimize blur, the natural depth of focus, pupil size variations on accommodation and eye dominance need to be used to provide the best resolved near and distance images, but no established diagnostic protocol exists to obtain this information from the individual. Therefore, success in fitting multifocal lenses is highly variable among eye care practitioners and success rates are, on average, less than about 52% over an average of 3.2 fitting visits.

### DETAILED DESCRIPTION OF THE INVENTION AND PREFERRED EMBODIMENTS

The invention provides methods for fitting multifocal contact lenses. The methods of the invention provide fitting of multifocal lenses that are less time consuming and result in more successful fittings than conventional methods.

In one embodiment, the invention provides a method for fitting multifocal contact lenses comprising, consisting essentially of, and consisting of: a.) assessing a potential for a successful multifocal lens fitting for an individual; b.) determining a dominant and a non-dominant eye for the individual; c.) measuring a manifest refraction for each eye of the individual; d.) determining an add power for the individual; e.) fitting a multifocal lens on each of the dominant and non-dominant eyes of the individual; and, optionally, f.) assessing lifestyle visual needs for the individual and refining the fit performed in step e.) for the dominant eye, the non-dominant eye, or both based on the assessment.

For purposes of the invention, by “dominant eye” is meant the eye that is determined by the eye care practitioner to be the eye the correction for which should be optimized for distance vision and the non-dominant eye refers to eye the correction for which should be optimized for near vision.

In the first step of the method of the invention, the potential for successful multifocal lens fitting for the individual is assessed. The purpose of the assessment is to identify those individuals who are unable to adapt to a multifocal lens as well as those who will not be satisfied with the visual performance of the lens. It is one discovery of the invention that visual satisfaction with a newly prescribed multifocal lens strongly correlates with the individual's satisfaction with his or her habitual vision correction. Additionally, four parameters were identified as most important in determining the visual satisfaction with the habitual correction, which factors are distance vision satisfaction, near vision satisfaction, overall vision satisfaction and glare perception. An index of satisfaction with habitual correction is:

S=f(D, N, O, G)

wherein D is distance vision satisfaction;
N is near vision satisfaction;
O is overall vision satisfaction; and
G is glare perception.

Each variable may be rated on a scale such as 1 to 5, with 1 being the lowest and 5 the highest. In such a scale, if the D, N, O, and G are summed, an S of 16 or more is high and 19 is very high. Individuals with high or very high S values are those unlikely to be successfully fitted because they are sufficiently satisfied with their habitual correction that it is unlikely that a benefit from being fitted with multifocal lenses will be perceived and such individuals are screened out from multifocal lens fitting. Thus, only individuals with a satisfaction index of less than 19 and preferably less than 16 are fitted with multifocal lenses.

Optionally, one or more variables may be added. For example, if the individual is highly motivated to wear multifocal lenses or they have dry eye symptoms, variables may be added into determining of the satisfaction index. As another alternative, other factors may be included such as disparity or anisometropic blur, between the eyes and comfort with wearing contact lenses. Additionally, the function may be refined based on the lifestyle assessment. For example, D and G may be weighted more heavily than N and O if the individual is truck driver. This may be reflected, for example, in the following equation:

S=W_{D}D+W_{N}N+W_{O}O+W_{G}G

wherein W_{D} is the weight on the distance score;
W_{N} is the weight on the near score;
W_{O} is the weight on the overall vision satisfaction; and
W_{G} is the weight on the glare perception score.

Still as another alternative, objective visual performance may be included. For example, individuals with visual acuity equal to or better than 20/25 at distance and 20/30 at near will be significantly less likely to be successfully fitted with new multifocal lenses.

An alternative or additional step for assessing the success potential is to assess blur tolerance by having the individual view a target at a distance, preferably a chart at about 20 feet while adding measured amounts of defocus, or plus power, to each eye in turn. More preferably, blur tolerance is measured at 20 feet and a near distance of about 40 cm. The measured individuals may be categorized by the blur response. For example, individuals may be categorized as tolerant to blur bilaterally, tolerant to blur unilaterally, sensitive to blur bilaterally or sensitive to blur unilaterally. Those individuals in either of the unilateral categories more likely will be successfully fitted for multifocal lenses than those in either of the two bilateral categories.

Once it is determined that the individual is to be fitted with multifocal lenses, subsequently, the dominant eye is determined, manifest refraction is measured, an add power is determined, and, optionally, lifestyle visual needs are assessed. The dominant eye may be determined by any convenient method, but preferably is determined by assessing the binocular blur tolerance as described above.

The manifest refraction, meaning the distance vision at infinity and near vision correction needed for what is comfortable reading vision, is measured without cycloplegia of the eyes. The measurement is carried out using any convenient method and equipment including, without limitation, use of a phoropter or aberrometer. Comfortable vision may be defined subjectively by the individual's response or objectively as by determining the distance at which the individual experiences binocular fusion and the image size is optimized against convergence needs.

The add power, meaning positive sphere power in addition to that required for distance correction, is determined by any convenient method. Preferably, the add power is determined using the binocular cross, or fused cross, cylinder.

Once manifest refraction is measured, increasing plus power is added to each eye while measuring the visual performance. Typically, individuals will prefer power that is more plus than the manifest refraction by an amount equal to one-half of the depth of focus. The depth of focus will vary with the physiology of the eye, corneal and crystalline lens aberrations and the eye's optic axis length. The range of additional plus power will be about 0.5 to about 1.5 diopters and typically will be about 0.5 diopters. It is important to achieve minimum image blur for both eyes at the same distance, unless the individual is being fitted for monovision or modified monovision, to minimize the anisometropic image blur and achieve best stereopsis.

The individual's dominant and non-dominant eyes are then fit with lenses. The dominant eye is fit with a multifocal lens that provides visual acuity correction substantially equal to the spherical equivalent, or sphero-cylindrical manifest refraction. The non-dominant eye is fit with a lens in accordance with the add power. Typically, the non-dominant eye will have less than about 0.5 diopters more plus power than the spherical equivalent for that eye.

After the individual is first fitted with the lenses, the lenses preferably are worn for some period while the individual remains in the practitioner's facilities to assess initial adaptation. The assessment may include one or more of over-refraction to ensure the lenses provide the intended correction, tolerance testing, image blur suppression testing, and subjective image quality testing. Thereafter, preferably the individual is reassessed in seven to ten days.

The fit on the dominant and non-dominant eyes may be, and preferably are, optimized for best subjective distance and near vision to take into account the lifestyle visual needs. Thus, in an optional step an assessment of the individual's lifestyle needs may be carried out and the lenses' fit optimized based on those needs. The assessment may be carried out by any convenient method including, without limitation, questioning of the individual directly or through the use of a questionnaire

The responses may be weighted and then added to provide a weighted score for determining the balance between distance and near vision needs. Alternatively, the responses may be grouped in two groups; one to modulate lens selection for the dominant eye and one for the non-dominant eye. Weighted scores may be obtained for each group and used to determine the first fit.

The fitting method of the invention may be used to fit a variety of multifocal lenses, but may find its greatest utility in the fitting of lenses from a set of three lenses, each lens having a power profile different from that of each of the other lenses and the lenses satisfying the following relationships:

D̅≧−0.14×Rx_add+0.84

N̅≧−0.08×Rx_add+0.64

Δ̅d≦0.2

Δ̅n≦0.2

wherein D̅ is a mean value of a binocular weighted distance ratio for pupil diameters from 2.5 to 6 mm;
Rx_add is the additional power in diopters added to the distance prescription to provide near vision correction for an individual;
N̅ is a mean value of a binocular weighted near ratio for pupil diameters from 2.5 to 6 mm;
Δ̅d is a mean value for a disparity at distance vision between the first and second lens for pupil diameters of about 2.5 to 6 mm; and
Δ̅n is a mean value for a disparity at near vision between the first and second lens for pupil diameters of about 2.5 to about 6 mm.

The binocular weighted distance ratio (“D”) is the maximum of the weighted distance ratio of the dominant eye (“d₁”) and the weighted distance ratio of the non-dominant eye (“d₂”) or D=max (d₁, d₂). The weighted near ratio (“N”) is the maximum of the weighted near ratio of the dominant eye (“n₁”) and the weighted near ratio of the non-dominant eye (“n₂”) or N=max (n₁, n₂).

The monocular weighted distance and near ratios may be calculated for various pupil sizes for each eye and are measures of how well the power at any given lens radius meets the distance and near requirements, respectively, of the lens wearer. The ratios also measure how well a single lens may be expected to perform relative to ideal given the wearer's sphere and add prescriptions. The weighted distance and near ratios will have a range of values from 0 to 1.0, with 0 meaning that no benefit is provided at the required distance for the lens wearer and 1.0 meaning that the lens fully corrects the wearer at the distance. For rotationally symmetric power profiles, the monocular weighted distance ratio may be calculated by integrating over the lens radius to give:

$$d ( R ) = \frac{{\int }_{0}^{R} [ 1 - tanh ( 0.5 ⋆ \vert P ( r ) - Rx\_sphere \vert ) ] r d r}{{\int }_{0}^{R} r d r} \qquad ( I )$$

wherein R is a radius of the pupil;
Rx_sphere is a sphere prescription power in diopters for the eye that the monocular weighted ratio is being calculated;
tanh is the hyperbolic tangent; and
P(r) is the power of the lens plus eye given by the following equation:

P(r)=P_{CL}(r)+SA_{eye}*r²+F (II)

wherein SA_{eye} is the spherical aberration of the eye and preferably is 0.1 diopters/mm²;
F is the lens fit, meaning the change from nominal, in diopters;
r is a radial distance from the center of the contact lens; and
P_{CL}(r) is the radial power distribution, or power profile, for the contact lens. For a specific design, the power distribution is provided as a series of P_{CL}(r) in increments of 0.25 diopters.

The radial power distribution, or power profile, (P_{CL}(r)) of the lens is the axial power of the lens in air and may be calculated from the surface shapes, thickness and index of refraction of the lens.

The monocular weighted near ratio may be calculated by integrating over the lens radius to give:

$$n ( R ) = \frac{{\int }_{0}^{R} [ 1 - tanh ( 0.5 ⋆ \vert \begin{matrix}P ( r ) - Rx\_sphere - \\ Rx\_add\end{matrix} \vert ) ] r d r}{{\int }_{0}^{R} r d r} \qquad ( III )$$

wherein R, is the radius of the pupil;
Rx_sphere is a sphere prescription power in diopters for the eye that the monocular weighted ratio is being calculated;
tanh is the hyperbolic tangent;
P(r) is the power of the contact lens plus eye given by Equation II; and
Rx_add is an additional power in diopters added to a distance prescription to provide near vision correction for the individual.

For non-rotationally symmetric power profiles, the monocular weighted distance ratio may be calculated by integrating over the lens radius to give:

$$d ( R ) = \frac{{\int }_{0}^{2 π} {\int }_{0}^{R} [ 1 - tanh ( 0.5 ⋆ \vert \begin{matrix}P ( r , Φ ) - \\ Rx\_sphere\end{matrix} \vert ) ] r d r d Φ}{{\int }_{0}^{2 π} {\int }_{0}^{R} r d r d Φ} \qquad ( IV )$$

wherein R, Rx_sphere, tanh and P(r) are as set forth above: and
Φ is a polar angle.

The monocular weighted near ratio for non-rotationally symmetric power profiles may be calculated by integrating over the lens radius to give:

$$n ( R ) = \frac{{\int }_{0}^{2 π} {\int }_{0}^{R} [ 1 - tanh ( 0.5 ⋆ \vert \begin{matrix}\begin{matrix}P ( r , Φ ) - \\ Rx\_sphere -\end{matrix} \\ Rx\_add\end{matrix} \vert ) ] r d r d Φ}{{\int }_{0}^{2 π} {\int }_{0}^{R} r d r d Φ} \qquad ( V )$$

For symmetrical diffractive lenses, the monocular weighted distance ratio may be calculated by integrating over the lens radius to give:

$$d ( R ) = \frac{{\int }_{0}^{R} [ 1 - tanh ( 0.5 ⋆ \vert {\sum }_{m} {ɛ}_{m} ⋆ {P}_{m} ( r ) - Rx \vert ) ] r d r}{{\int }_{0}^{R} r d r} \qquad ( VI )$$

wherein m is the diffractive order;
Pₘ(r) is the power profile into order m;
εₘ is the diffractive efficiency into order m; and

$${\sum }_{m} {ɛ}_{m} is 1.$$

Equations II, IV and V may be similarly modified.

For purposes of the invention, by “a set of three lenses” is not meant literally only three lenses, but rather three subsets of lenses each of which subsets is composed of multiple lenses that provide sphere power and add power over desired ranges. Preferably, each subset is composed of multiple lenses that provide sphere power over the range of −12.00 to +8.00 diopters in 0.25 diopters increments and add power over the ranges of 0.75 to 2.50 diopters in increments of 0.25 diopters. More preferably, one subset of lenses provides sphere power over the range of −12.00 to +8.00 diopters in 0.25 diopters increments and add power over the ranges of 0.75 to 1.75 diopters in increments of 0.25 diopters, a second subset of lenses provides sphere power over the range of −12.00 to +8.00 diopters in 0.25 diopters increments and add power over the ranges of 0.75 to 2.50 diopters in increments of 0.25 diopters, and a third subset of lenses provides sphere power over the range of −12.00 to +8.00 diopters in 0.25 diopters increments and add power over the ranges of 1.25 to 2.50 diopters in increments of 0.25 diopters.

Still more preferably, the method the invention is used in conjunction with the fitting of lenses from a set of three lenses, each lens having a power profile different from that of each of the other lenses and the lenses satisfying the following relationships:

D̅≧−0.14×Rx_add+0.84

N̅≧−0.08×Rx_add+0.64

Δ̅d≦0.2

Δ̅n≦0.2

wherein the front surface, or object side surface, of the lens is a zone multifocal surface or a continuous aspheric multifocal surface and the back surface, or eye side surface, of the lens is an aspheric surface. By “zone multifocal surface” is meant that there is a discontinuity as one moves from one power zone to another power zone. The aspheric back surface preferably has a radius of approximately 7.20 to 8.10 mm and more preferably 7.85 mm, from the geometric center to the lens edge and a conic constant of −0.26.

In a still more preferred embodiment, the fitting method of the invention is used to fit lenses having a front multifocal surface with five, radially symmetric zones that alternate between near correction and distance correction or near, distance and intermediate correction and an aspheric back surface with a radius of approximately 7.20 to 8.10 mm and more preferably 7.85 mm, and a conic constant of −0.26. In Table 2 below provides more preferred values for the set of three lenses, A, B, and C within this embodiment.

TABLE 2

| | A | B | C |
|---|---|---|---|
| Nominal Zone Height | 0.6 | 0.9 | 1.9 |
| (diopters) | | | |
| Zone Height Range | 0.3 to 0.8 | 0.7 to 1.2 | 1.7 to 2.1 |
| Spherical Aberration | −0.1 | −0.17 | −0.1 |
| (diopters/mm²) | | | |
| Spherical Aberration | −0.08 to −0.12 | −0.14 to −0.20 | −0.8 to −0.12 |
| Range | | | |
| Zone Transitions-1^{st} | 0.75 | 0.7 | 1 |
| Zone Transitions-1^{st} | 0.65 to 0.85 | 0.6 to 0.8 | 0.9 to 1.1 |
| Range | | | |
| Zone Transitions-2d | 1.25 | 1.3 | 1.95 |
| Zone Transitions-2d | 1.15 to 1.35 | 1.2 to 1.4 | 1.85 to 2.05 |
| Range | | | |
| Zone Transitions-3^{rd} | 2 | 1.95 | 2.5 |
| Zone Transitions-3^{rd} | 1.9 to 2.1 | 1.85 to 2.05 | 2.4 to 2.6 |
| Range | | | |
| Zone Transitions-4^{th} | 2.5 | 2.55 | 3.45 |
| Zone Transitions-4^{th} | 2.4 to 2.6 | 2.45 to 2.65 | 3.35 to 2.55 |
| Range | | | |

In a yet more preferred embodiment, the fitting method of the invention is used to fit a set of three lenses, each lens having a power profile different from that of each of the other lenses and the lenses satisfying the following relationships:

D̅≧−0.14×Rx_add+0.84

N̅≧−0.08×Rx_add+0.64

Δ̅d≦0.2

Δ̅n≦0.2

wherein the front surface is a zone multifocal surface in which in each zone is incorporated spherical aberration in which spherical aberration of the near zones may be an additional plus or minus 0.05 to 0.2 diopters/mm² from that of the distance zones.

Alternatively, whether the multifocal surface is a continuous or discontinuous surface, the spherical aberration for distance and near may be adjusted according to the following equations:

SA_{RX}=SA₀+c*Rx_sphere

0.0044<c<0.0052

wherein SA₀ is the spherical aberration of the design for an Rx_sphere that equals 0.0 diopters;
c is a constant of a value between 0.0044 and 0.0052 and preferably is 0.0048. The back surface of the lens in these embodiments is preferably aspheric with a radius of approximately 7.20 to 8.10 mm, more preferably 7.85 mm and a conic constant of −0.26.

In yet another embodiment of the invention, the fitting method is used to fit a set of three lenses, each lens having a power profile different from that of each of the other lenses and the lenses satisfying the following relationships:

D̅≧−0.14×Rx_add+0.84

N̅≧−0.08×Rx_add+0.64

Δ̅d≦0.2

Δ̅n≦0.2

STD(P_{E}(r))<0.15 for 1.25<r<3.

wherein STD is the standard deviation; and
P_{E}(r) is the effective lens plus-eye power given by the following equation:

$${P}_{E} ( r ) = {\int }_{0}^{R} P ( r ) ⋆ r d r \qquad ( VI )$$

wherein P(r) is the power of the contact lens on the eye given by:

P(r)=P_{CL}(r)+SA_{eye}*r²+F (VIII)

wherein SA_{eye} is the spherical aberration of the eye and preferably is 0.1 diopters/mm²;
F is the lens fit, meaning the change from nominal, in diopters;
r is a radial distance from the center of the contact lens; and
P_{CL}(r) is the radial power distribution, or power profile, for the contact lens. For a specific design, the power distribution is provided as a series of P_{CL}(r) in increments of 0.25 diopters.

In the zone designs used with the fitting method of the invention, the first zone, or the zone that is centered at the geometric center of the lens may be, and preferably is, a zone that provides distance vision correction or it may provide near or intermediate vision correction. In lens pairs, the first zone may be the same or different. Similarly, in continuous, aspheric multifocal designs, the correction at the center of each of the lens pairs may be the same or different and may be selected from distance, intermediate and near correction.

Contact lenses that may be used in the fitting method of the invention preferably are soft contact lenses. Soft contact lenses, made of any material suitable for producing such lenses, preferably are used. Illustrative materials for formation of soft contact lenses include, without limitation silicone elastomers, silicone-containing macromers including, without limitation, those disclosed in U.S. Pat. Nos. 5,371,147, 5,314,960, and 5,057,578 incorporated in their entireties herein by reference, hydrogels, silicone-containing hydrogels, and the like and combinations thereof. More preferably, the surface is a siloxane, or contains a siloxane functionality, including, without limitation, polydimethyl siloxane macromers, methacryloxypropyl polyalkyl siloxanes, and mixtures thereof, silicone hydrogel or a hydrogel, such as etafilcon A.

A preferred lens-forming material is a poly 2-hydroxyethyl methacrylate polymers, meaning, having a peak molecular weight between about 25,000 and about 80,000 and a polydispersity of less than about 1.5 to less than about 3.5 respectively and covalently bonded thereon, at least one cross-linkable functional group. This material is described in U.S. Pat. No. 6,846,892 incorporated herein in its entirety by reference. Suitable materials for forming intraocular lenses include, without limitation, polymethyl methacrylate, hydroxyethyl methacrylate, inert clear plastics, silicone-based polymers, and the like and combinations thereof.

Curing of the lens forming material may be carried out by any means known including, without limitation, thermal, irradiation, chemical, electromagnetic radiation curing and the like and combinations thereof. Preferably, the lens is molded which is carried out using ultraviolet light or using the full spectrum of visible light. More specifically, the precise conditions suitable for curing the lens material will depend on the material selected and the lens to be formed. Polymerization processes for ophthalmic lenses including, without limitation, contact lenses are well known. Suitable processes are disclosed in U.S. Pat. No. 5,540,410 incorporated herein in its entirety by reference.

What is claimed is:

1. A method for fitting multifocal contact lenses, comprising the steps of: a.) assessing a potential for a successful multifocal lens fitting for an individual; b.) determining a dominant and a non-dominant eye for the individual; c.) measuring a manifest refraction for each eye of the individual; d.) determining an add power for the individual; e.) fitting a multifocal lens on each of the dominant and non-dominant eyes of the individual.

2. The method of claim 1, further comprising f.) assessing lifestyle visual needs for the individual and refining the fit performed in step e.) for the dominant eye, the non-dominant eye, or both based on the assessment.

3. The method of claim 1, wherein step a.) comprises calculating an index of satisfaction with habitual correction for an individual.

4. The method of claim 1, wherein step a.) comprises assessing blur tolerance for each eye.

5. The method of claim 3, wherein step a.) further comprises assessing blur tolerance for each eye.

6. The method of claims 3 or 4, further comprising categorizing the individual as tolerant to blur bilaterally, tolerant to blur unilaterally, sensitive to blur bilaterally or sensitive to blur unilaterally.

7. The method of claim 2, wherein step f.) further comprises providing a weighted score for determining the balance between a distance vision and a near vision need for the individual.

8. The method of claim 2, wherein step f.) further comprises grouping assessment responses into a first group to modulate lens selection for the dominant eye and a second group for the non-dominant eye and obtaining weighted assessment scores for each group.
